(19) United States
(12) Patent Application Publication
(10) Pub. No.: US 2007/0282163 A1
(43) Pub. Date: Dec. 6, 2007

(54) Penis enhancement and exercise device

(76) Inventors: Howard J. Williams, Power Springs, GA (US); Stephen M. Frazee, Marietta, GA (US)

Correspondence Address: Harry I. Leon, 924 Bowen St. NW, Atlanta, GA, 30318, US

(21) Appl. No.: 11/373,647

(22) Filed: Mar. 10, 2006

Related U.S. Application Data
(60) Provisional application No. 60/691,347, filed on Jun. 20, 2005.

(51) Int. Cl. A61F 5/00

(52) U.S. Cl. 600/41

(57) ABSTRACT

A short annular body for supporting the base of the penis during Jelq and other exercises to strengthen and enlarge this organ. The annular body, formed of a rubbery material, has rounded ends and a beveled portion disposed symmetrically about a central opening. Juxtaposed between the rounded ends, the beveled portion has tapered inner and outer surfaces, which diverge slightly from each other, reaching their greatest divergence midway along the axial length of the annular body. Prior to use, the consumer slides the annular body along the flaccid penis until the beveled portion contacts the crotch. With the annular body so positioned, the base of the penis is supported and ready for exercise. Since the user no longer has to hold its base with his fingers, he can then stroke the penis along its full length without interference from the holding hand, providing for more comfortable and efficient exercise.

### CROSS REFERENCE TO RELATED APPLICATION

The subject matter of this application was disclosed in U.S. Provisional Patent Application Ser. No. 60/691,347, filed Jun. 20, 2005.

### FIELD OF THE INVENTION

This present invention relates generally to sexual aids and more particularly is a device to aid in exercises to strengthen and enlarge the penis.

### BACKGROUND OF THE INVENTION

Many men encounter problems such as the inability to maintain an erection or premature ejaculation. Discussing these or other concerns such as the size of a penis can be embarrassing. With only the slim hope of combating these difficult, personal issues, many men choose not to seek medical assistance or risk taking prescription drugs which have serious side effects, including increasing the blood pressure. Men would be much happier if they could resolve these troubles in the comfort of their own homes in a safe and confidential manner.

### SUMMARY OF THE INVENTION

The object of this invention there is to provide a device which can be slid onto a non-aroused penis to aid in penis enlargement and strengthening exercise, so that a firm erection can be maintained during intercourse and premature ejaculation can be prevented.

In accordance with the present invention, there is provided a penis enhancement and exercise device (hereinafter “the device”) comprising a short annular body which defines a central hole sized to slideably receive the flaccid penis of a typical adult human male. Disposed generally symmetrically about the central hole are the first and second rounded ends and a beveled portion. Preferably fabricated from a rubbery material, they form a single, unitary piece with the beveled portion juxtaposed between the rounded ends and extending about one-half the axial length of the annular body. Outer and inner surfaces on the beveled portion taper outwardly and inwardly, respectively, in the direction of the juncture between the first rounded end and the beveled portion, creating a mid-section bulge. The comfort level of the wearer is substantially enhanced when the annular body is oriented in such a way that the second rounded end and the beveled portion face the crouch.

Designed to be used in combination with well-know exercises, which entail light slow stroking, pulling and twisting motions applied manually to a flaccid penis, the device makes such exercises far more effective and relaxing. Aware that an erection occurs when certain chambers of the penis fill with blood, exercise practitioners, whether aided by the device or otherwise, attempt, through numerous repetitions, to maximize penis size by causing the cells of the erectile tissue to expand by forcing more blood into them. Specifically, situated within and atop the penis structure and known medically as the Corpora Cavernosa are two large blood holding chambers which together store about 90 percent of the blood every time a penis erection occurs. These blood holding chambers are the primary targets of a concerted effort in which exercise practitioners try to break down their cell walls, by forcing more blood into the cells. After exercise, the cell walls will grow back, larger and stronger, as the penis tries to heal itself. Without the device, the user must support the base of the penis with one hand while each exercise is performed using the other hand. With the device, the base of the penis is supported by the device held between the thumb and middle finger of the user's hand, Since the device is only about one-half inch thick, it allows nearly full length stroking of the penis with the other hand. This allows a more efficient exercise of the penis and reduces the time needed for each workout.

A typical example of the device in use involves the standard penis enhancement exercise known as the Jelq method. In this exercise, the device is slipped over the non-erect penis with the narrow end of the annular body toward the crotch. As one hand gently presses the annular body against the crotch, the fingers of the other hand encircle the penis and slowly move from the base of the penis to its head in a smooth, gentle milking motion. This milking motion is performed along virtually the entire length of the penis from the base to the tip. In the original Jelq exercise, the base of the penis must be held by the fingers of the second hand while stroking is done with the other. The Jelq method is enhanced with the device because it allows for full length stroking without interference by the holding hand and provides for a more comfortable and efficient exercise.

### BRIEF DESCRIPTION OF THE DRAWINGS

FIG. 1 is a top side perspective view of the penis enhancement and exercise device according to the present invention;

FIG. 2 is a bottom side perspective view of the device according to FIG. 1;

FIG. 3 is a top plan view of the device according to FIG. 1;

FIG. 4 is a bottom plan view of the device according to FIG. 1;

FIGS. 5 and 6 are side elevational views of fragmentary portions of the device according to FIG. 1, showing outer and inner surfaces, respectively, of the device; and

FIG. 7 shows the device according to FIG, 1 and its position mounted on the penis and being used by a consumer to perform the Jelq exercise.

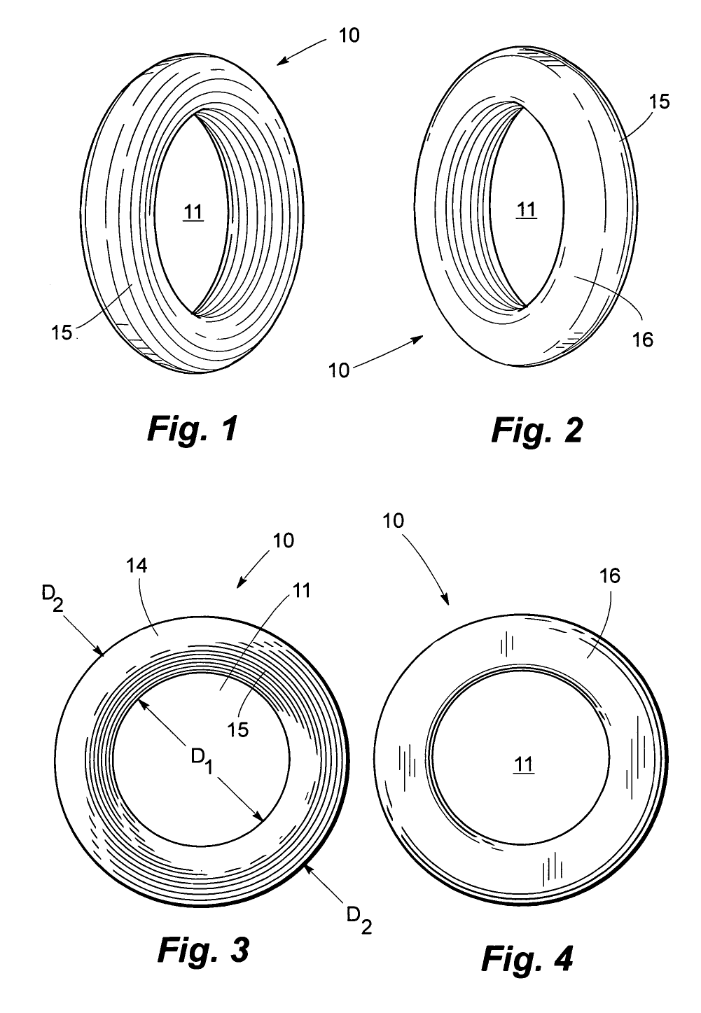

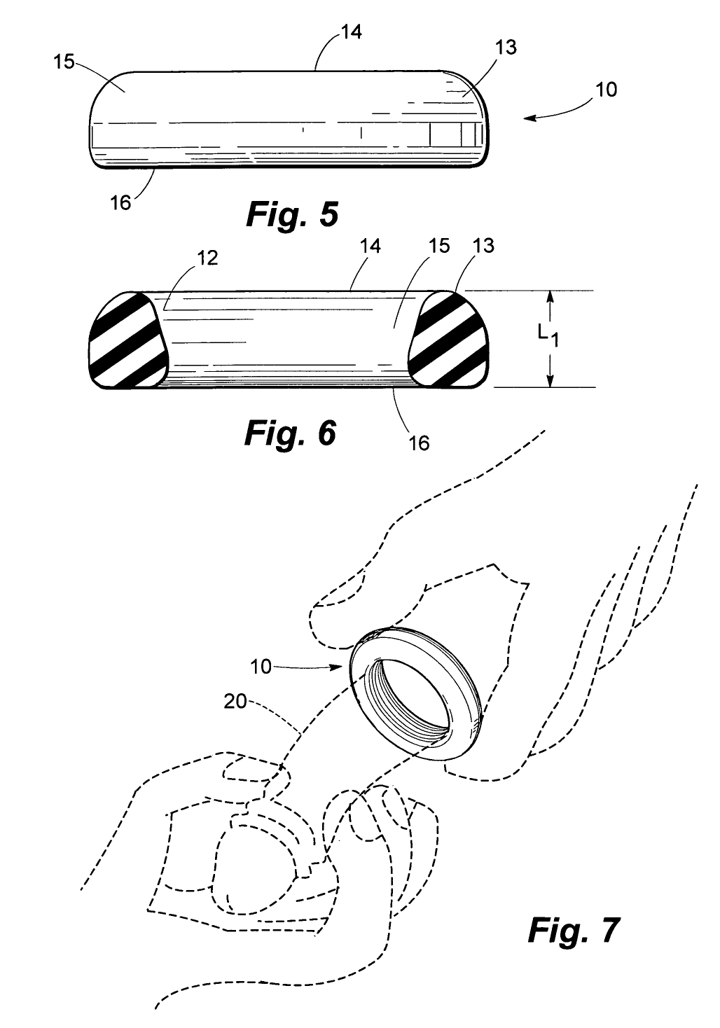

### DETAILED DESCRIPTION OF THE PREFERRED EMBIMENT

In the drawings, a penis enhancement and exercise device is indicated generally by the reference numeral 10. The device 10, preferably formed as a monolithic, annular structure with a central hole 11, is sized to fit the flaccid male organ 20 (FIG. 7). Inner and outer surfaces of the annular structure are tapered for about one-half of its axial length, giving rise to chamfers 12, 13, respectively, which extend away from rounded base 14. The chamfers 12, 13 facilitate mounting the device 10 and add to- a user's comfort.

Between the extremities of rounded ends 14, 16, the device 10 has sufficient axial length that in use one can hold it between the thumb and index finger (FIG. 7). The axial length of the device 10 preferably measures about ½ inch.

For most men, as they embark on penis enlargement and strengthening exercises using the device 10, such a device with a central hole 11 whose inner diameter D₁ measures between 1¼ and 1½ inches has been found to be satisfactory. As the consumer achieves desired penis enlargement results over time, he will preferably opt to utilize devices 10 with increasingly larger diameters D₁.

Fabricated as a single, unitary piece of a rubbery material, such as polyurethane elastomers or synthetic rubber, and having a preferred hardness in the range of 50 to 65 durameters, the annular structure has sufficient thickness, in transverse cross-section, that the annular structure generally retains its shape when the inner and outer chamfers 12, 13 of beveled portion 15 are pinched together manually. In the preferred embodiment having an inner diameter D₁ which measures approximately 1¼ inches, the outer diameter D₂ of the device 10 at its greatest transverse cross-section measures, by way of example, about 2.0 inches. A suitable rubbbery material is available commercially from Santoprene.

What is claimed:

1. A device for aiding in exercises which strengthen and elongate the penis, comprising a short annular body which defines a central hole sized to slideably receive the flaccid penis of a typical adult human male, the annular body having first and second rounded ends and a beveled portion, the rounded ends and the beveled portion being disposed generally symmetrically about the central hole, the beveled portion being juxtaposed between the first and second rounded ends and being joined thereto; the first rounded end having a radius of curvature which is equal to about one-half the thickness, in transverse cross-section, of the annular body at the juncture between the first rounded end and the beveled portion; the beveled portion having outer and inner surfaces which taper outwardly and inwardly, respectively, in the direction of said juncture; the rounded ends and the beveled portion forming a single, unitary piece.

2. The device according to claim 1 wherein the annular body is further characterized as being formed of a rubbery material and having sufficient thickness, in transverse cross-section, that the annular body substantially retains its shape when the outer and inner surfaces of the beveled portion are pinched together manually.

3. The device according to claim 2, wherein the rubbery material is further characterized as having a preferred hardness in the range of 50 to 65 durameters

4. The device according to claim 1 wherein the annular body is further characterized as having an axial length of about ½ inch and inner and outer diameters at said juncture of about 1¼ and 2.0 inches, respectively:
